(19) United States
(12) Patent Application Publication
(10) Pub. No.: US 2025/0011393 A1
(43) Pub. Date: Jan. 9, 2025

(54) FLAVONOID-COLLAGEN CROSSLINKED ANTIOXIDANT HYDROGEL

(71) Applicant: North Carolina State University, Raleigh, NC (US)

(72) Inventors: Danielle Nalband, Raleigh, NC (US); Donald O. Freytes, Raleigh, NC (US)

(21) Appl. No.: 18/760,168

(22) Filed: Jul. 1, 2024

Related U.S. Application Data
(60) Provisional application No. 63/512,297, filed on Jul. 7, 2023.

(51) Int. Cl. C07K 14/78; A61K 38/00; C08J 3/24

(52) U.S. Cl. CPC C07K 14/78; C08J 3/24; A61K 38/00; C08J 2389/00

(57) ABSTRACT

In one aspect, the disclosure relates to compositions including a collagen hydrogel and a flavonoid, wherein at least a portion of the collagen hydrogel is crosslinked by the flavonoid, methods of making the cosmetic and pharmaceutical compositions comprising the same, and methods of healing wounds using the same. The disclosed compositions are cytocompatible and can protect cells from oxidative damage through preventing damage from and/or reducing accumulation of reactive oxygen species (ROS). This abstract is intended as a scanning tool for purposes of searching in the particular art and is not intended to be limiting of the present disclosure.

### CROSS REFERENCE TO RELATED APPLICATIONS

This application claims the benefit of U.S. Provisional Application No. 63/512,297 filed on Jul. 7, 2023, which is incorporated herein by reference in its entirety.

### BACKGROUND

Wound healing is a natural and complex process consisting of a concerted series of biological events that aims to restore tissue integrity and function following an injury. In healthy individuals, this process is highly efficient and will complete within 4-6 weeks, excluding tissue remodeling. However, not all wounds will heal without complication, and the healing trajectory can be disrupted and prolonged due to age-related pathologies, obesity, and diabetes. Globally, approximately 40 million people suffer from chronic wounds, and studies have predicted that 1-2% of populations in developed countries will be afflicted with chronic wounds in their lifetime.

Cellular response to injury is regulated, in part, through appropriate redox signaling of reactive oxygen species (ROS) generated during injury and wound repair. However, excess ROS has been shown to have detrimental effects on healing. In chronic wounds, sustained inflammation can accumulate high levels of ROS at the wound site, overwhelming the tissue's natural ability to maintain a physiologically proficient redox environment and thus prolonging the healing process while inducing higher amounts of cellular oxidative damage and death.6 Conversely, the presence of low levels of ROS and the prevention of the accumulation of excess ROS at the site of injury has been shown by numerous studies to facilitate healing by killing pathogens, preventing infection, and activating cell migration and angiogenesis.

Selecting the appropriate wound care strategy is an important factor during the treatment of any wound to maximize successful patient outcomes. Considering chronic and complicated cutaneous wounds, collagen-based hydrogels are an attractive option since they establish a moist environment over the wound area while absorbing exudates, protect the wound bed, prevent bacterial infiltration, and allow gas exchange. Additional beneficial properties of collagen include its natural biological origin, inherent cytocompatibility, biodegradation rate, and non-toxic degradation products. Furthermore, the collagen scaffold can mimic the physiological extracellular matrix structure and can be combined with a cellular component to further harness the native secretory and regenerative abilities of versatile skin and stem cells for the repair of damaged tissue. Currently, there are several collagen-based commercial grafts on the market that utilize autologous or allogenic dermal fibroblasts cultured within the matrix to support skin regeneration including Apligraf® (available from Sandos A G, Basel, Switzerland), OrCel® (available from Orcell, LLPC, New York, NY), PermaDerm® (available from Regenicin, Inc., Little Falls, NJ), and StrataGraft® (available from Stratatech Corporation, Madison, WI). Overall this therapeutic approach could represent an alternative to costly autologous graft and eliminates the need for donor tissue in large wound coverage.

Acceleration of healing in chronic wounds could be achieved through strategic modulation of the microenvironment of the injury. Improving wound healing through antioxidant therapy has been recognized through oral supplements and topical products, including modified hydrogels. These materials, combining antioxidant species of natural and synthetic origin, have demonstrated potential to neutralize oxidant species, maintain the natural physiological balance of ROS during healing, and enhance wound healing responses, albeit almost exclusively outside of a clinical setting. Yet, novel formulations to deliver bioprotective antioxidants in tandem with cell-based therapies represents untapped therapeutic potential. These materials could redirect the body's response in chronic wounds from stagnation at early stages of wound repair toward progressive healing by directly influencing the chemical and cellular environment at the wound site. Few antioxidant therapeutic modalities for cutaneous wound healing have progressed to clinical trials, and none have combined natural, exogenous antioxidants with a cell-based therapy in one material.

Previous studies have established luteolin, an abundant flavonoid, as a multi-functional, biologically active molecule with reported anti-inflammatory, antioxidant, and immunomodulatory activity. Therefore, herein, it is broadly hypothesized that the incorporation of luteolin, together with a primary dermal fibroblast cell line, within a collagen hydrogel matrix could act synergistically with the body's endogenous mechanisms for effective wound treatment, potentially changing the outlook, prognosis, and treatment of difficult and complicated wounds (FIG. 1).

Despite advances in wound healing and treatment research, there is still a scarcity of hydrogel compositions that are cytocompatible, that have antioxidant activity and are effective at reducing and/or preventing accumulation of reactive oxygen species, and have desirable physical properties. These needs and other needs are satisfied by the present disclosure.

### SUMMARY

In accordance with the purpose(s) of the present disclosure, as embodied and broadly described herein, the disclosure, in one aspect, relates to compositions including a collagen hydrogel and a flavonoid, wherein at least a portion of the collagen hydrogel is crosslinked by the flavonoid, methods of making the cosmetic and pharmaceutical compositions comprising the same, and methods of healing wounds using the same. The disclosed compositions are cytocompatible and can protect cells from oxidative damage through preventing damage from and/or reducing accumulation of reactive oxygen species (ROS).

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows (top) overview of the process by which excessive accumulation of reactive oxygen species (ROS) at a wound site can lead to a stagnation in the healing progression and result in chronic wounds. Engineered wound healing materials formulated with antioxidants to reduce the amount of ROS at the wound site and also deliver viable, undamaged cells to proliferate, secrete collagen, and progress the closure and healing of the wound present a potential solution; (bottom): schematic showing how antioxidant, collagen-based hydrogels as are bioprotective materials against ROS in an in vitro oxidative environment compared to collagen hydrogels. Formulated with luteolin, these gels do not only modulate ROS levels around the wound site but also prevent ROS damage to live fibroblasts within the matrix.

FIGS. 2A-2D show physical characterization of luteolin-collagen hydrogels disclosed herein. (FIG. 2A) Image of inversion test showing self-standing gels using collagen hydrogels formulated with increasing concentrations of luteolin (0-120 M) or formulated using only vehicle, ethanol, (EtOH). (FIG. 2B) Normalized turbidity assay conducted using absorbance measurements at 405 nm at 37° C. over 90 minutes. (FIG. 2C) Frequency dependence of the elastic (G′) and viscous (G″) moduli and (FIG. 2D) G′ at 1 rad/s for collagen samples with increasing concentrations of luteolin (0-120 μM) and ethanol (EtOH) at 37° C.

FIGS. 3A-3C show cryoSEM Imaging and Pore Size Analysis of luteolin-collagen scaffolds. Representative cryoSEM images (Top) and Corresponding Histograms of Pore Size Distribution (Bottom). (FIG. 3A) 0 μM (FIG. 3B) 80 M (FIG. 3C) 120 μM.

FIGS. 4A-4D show cytocompatibilty studies. (FIG. 4A) Live/Dead Assay Live Cell Count Results, quantified using FIJI (NIH, USA), for normal adult human fibroblasts (NADFb) grown on 0-120 μM hydrogels after 48 hours in culture. Error bars represent StDev. n=4. Statistical significance: * p<0.05; ** p<0.01; *** p<0.001; **** p<0.0001. (FIG. 4B) Representative Live/Dead images of NADFb cultures after 48 hours. (FIG. 4C) Representative MTT Assay measuring metabolic activity of NADFb after 48 hours in 3D culture. Error bars represent StDev. n=3. (FIG. 4D) Comparison of the percent cell viability from Live/Dead and metabolic activity from MTT. Data are represented as a precent normalized to 0 μM samples.

FIGS. 5A-5B show antioxidant capacity of Luteolin-Collagen Hydrogels. (FIG. 5A) Total antixodiant activity of luteolin-collagen pre-gel solution was determined relatived to Trolox equivalents per mg of sample material. n=6. Statistical significance: * p<0.05; *** p<0.001. n=3. (FIG. 5B) Demonstrated antioxidant protection of NADFbs in 3D culture within luteolin-collagen hydrogels against 300 μM H₂O₂ exposure for 2 hours determined by MTT assay relative to the 0 μM control untreated with H₂O₂. Statistical significance: ** p<0.01; *** p<0.001; **** p<0.0001. n=3.

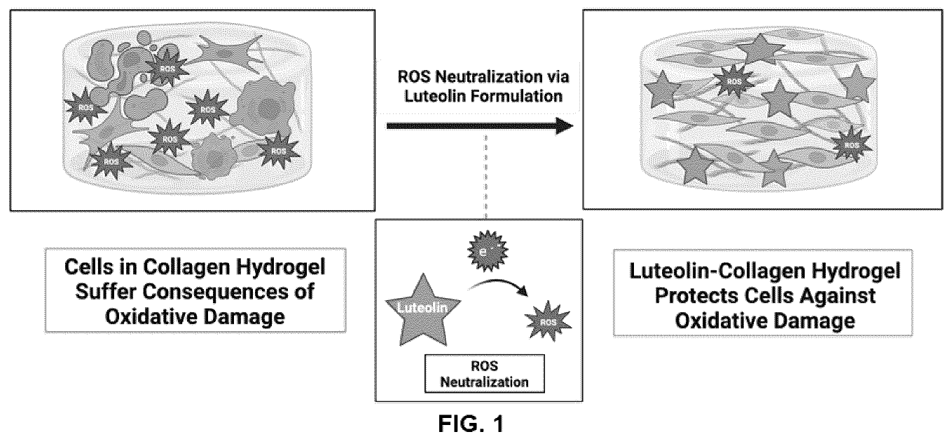

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are collagen hydrogels combined with luteolin and/or other flavonoids. In one aspect, the disclosed collagen hydrogels provide superior antioxidant effects in vitro. In another aspect, the disclosed collagen hydrogels can serve as a delivery system and protective environment for dermal fibroblasts. In another aspect, the disclosed hydrogels are cytocompatible and have the physical, chemical, and biologically active properties of a tissue regenerative material. In one aspect, the disclosed hydrogels can be formulated with luteolin and possess superior antioxidant activity compared to standard collagen. In another aspect, the disclosed hydrogels can be used to reduce and/or prevent accumulation of reactive oxygen species (ROS) at wound sites, thereby protecting cells against excessive oxidative damage. In an aspect, the addition of cultured primary dermal fibroblasts within the hydrogel matrix can localize the amount of viable, restorative cells at wound sites and can amplify the healing potential of these proposed therapeutics in an ROS-stabilized environment.

### Collagen Hydrogels Crosslinked by Flavonoids

In one aspect, disclosed herein is a composition including at least a collagen hydrogel and a flavonoid, wherein at least a portion of the collagen hydrogel is crosslinked by the flavonoid. In one aspect, at least 1% of the collagen hydrogel is crosslinked by the flavonoid. In one aspect, at least 5% of the collagen hydrogel is crosslinked by the flavonoid. In one aspect, at least 10% of the collagen hydrogel is crosslinked by the flavonoid. In one aspect, at least 15% of the collagen hydrogel is crosslinked by the flavonoid. In one aspect, at least 20% of the collagen hydrogel is crosslinked by the flavonoid. In one aspect, at least 25% of the collagen hydrogel is crosslinked by the flavonoid. In one aspect, at least 30% of the collagen hydrogel is crosslinked by the flavonoid. In one aspect, at least 35% of the collagen hydrogel is crosslinked by the flavonoid. In one aspect, at least 40% of the collagen hydrogel is crosslinked by the flavonoid. In one aspect, at least 45% of the collagen hydrogel is crosslinked by the flavonoid. In one aspect, at least 50% of the collagen hydrogel is crosslinked by the flavonoid. In one aspect, at least 55% of the collagen hydrogel is crosslinked by the flavonoid. In one aspect, at least 60% of the collagen hydrogel is crosslinked by the flavonoid. In one aspect, at least 65% of the collagen hydrogel is crosslinked by the flavonoid. In one aspect, at least 70% of the collagen hydrogel is crosslinked by the flavonoid. In one aspect, at least 75% of the collagen hydrogel is crosslinked by the flavonoid. In one aspect, at least 80% of the collagen hydrogel is crosslinked by the flavonoid. In one aspect, at least 85% of the collagen hydrogel is crosslinked by the flavonoid. In one aspect, at least 90% of the collagen hydrogel is crosslinked by the flavonoid. In one aspect, at least 95% of the collagen hydrogel is crosslinked by the flavonoid. In one aspect, at least 99% of the collagen hydrogel is crosslinked by the flavonoid. In one aspect, substantially all of the collagen hydrogel is crosslinked by the flavonoid. It is further understood that “at least a portion” does not include compositions where none or substantially none of the collagen hydrogel is crosslinked by the flavonoid.

In another aspect, numerous flavonoids are contemplated and should be considered disclosed. In one aspect, the flavonoid can have one or more free hydroxyl groups in order to complete the crosslinking. In another aspect, the flavonoid can be selected from a flavonol, flavone, anthocyanidin, isoflavonoid, neoflavonoid, an anthoxanthin, a derivative thereof, or any combination thereof. In a further aspect, the derivative can be a natural derivative or a semisynthetic derivative.

In one aspect, the flavonol can be selected from catechin, gallocatechin, catechin 3-gallate, gallocatechin 3-gallate, epicatechin, epigallocatechin, epicatechin 3-gallate, epigallocatechin 3-gallate, leucoanthocyanidin, a derivative thereof, or any combination thereof.

In another aspect, the anthocyanidin can be selected from anthocyanidin, cyanidin, delphinidin, malvidin, pelargonidin, peonidin, petunidin, a derivative thereof, or any combination thereof.

In another aspect, the isoflavonoid can be selected from genistein, daidzein, glycitein, equol, lonchocarpan, laxiflorane, a derivative thereof, or any combination thereof.

In still another aspect, the neoflavonoid can be selected from calophylloid, dalbergichromene, coutareagenin, dalbergin, nivetin, a derivative thereof, or any combination thereof.

In yet another aspect, the anthoxanthin can be selected from quercetin, betaxanthin, canthaxanthin, a derivative thereof, or any combination thereof.

In one aspect, the flavone can be selected from primuletin, chrysin, tetochrysin, primetin, apigenin, acacetin, genkwanin, echoidinin, baicalein, oroxylin A, negletein, norwogonin, wogonin, liquiritigenin, naringenin, geraldone, tithonine, luteolin, 6-hydroxyluteolin, chrysoeriol, diosmetin, pillion, velutin, norartocarpetin, artocarpetin, scutellarein, hispidulin, sorbifolin, pectolinarigenin, cirsimaritin, mikanin, isoscutellarein, zapotinin, zapotin, cerrosillin, alnetin, tricetin, tricin, corymbosin, nepetin, pedalitin, nodifloretin, jaceosidin, cirsiliol, eupatorine, sinensetin, hypolaetin, onopordin, wightin, nevadensin, xanthomicrol, tangeretin, serpyllin, sudachitin, acerosin, hymenoxin, gardenin D, nobiletin, scaposin, a derivative thereof, or any combination thereof. In one aspect, the flavone is luteolin.

In another aspect, various collagen compounds are contemplated and should be considered disclosed for use in the disclosed compositions. In an aspect, the collagen hydrogel can include type I collagen, type II collagen, type III collagen, type IV collagen, type V collagen, type VI collagen, type VII collagen, type VIII collagen, type IX collagen, type XI collagen, type XXI collagen, or any combination thereof. In another aspect, the collagen can include bovine collagen, porcine collagen, chicken collagen, turkey collagen, egg collagen, fish collagen, recombinant collagen produced by bacterial fermentation, extracellular matrix (ECM)-derived collagen, or any combination thereof. In an aspect, when the collagen is produced by bacterial fermentation, it can be produced using a gene sequence derived from a human or other mammal, poultry, or marine source. In one aspect, the collagen can be a combination of type I collagen and type III collagen. Exemplary methods for production of acellular ECM collagen are provided in US Pre-Grant Publication 2023/0181796 which is incorporated by reference herein in its entirety.

In any of these aspects, the disclosed compositions are cytocompatible and can protect cells with which they are in contact from oxidative damage.

### Components and Properties of the Crosslinked Hydrogels

In one aspect, the composition includes from about 5 μM to about 400 μM of the flavonoid, from about 40 μM to about 120 μM of the flavonoid, or from about 80 to about 120 μM, about 100 to about 120 μM, or about 40, 50, 60, 70, 80, 90, 100, 110, or about 120 μM of the flavonoid, or a combination of any of the foregoing values, or a range encompassing any of the foregoing values.

In another aspect, the collagen hydrogel has an average pore size of from about 0.5 μm to about 10 μm, about 0.5 μm to about 8 μm, 0.5 μm to about 3 μm, about 1 μm to about 3 μm, or about 1 μm to about 2 μm, or of about 0.5, 1, 1.5, 2, 2.5, or about 3 μm, or a combination of any of the foregoing values, or a range encompassing any of the foregoing values.

In another aspect, the collagen hydrogel has a Trolox equivalent antioxidant capacity (TEAC) value of at least about 2 mM/mg. In still another aspect, the collagen hydrogel retains at least about 45% metabolic activity relative to an untreated control after exposure to 300 μM hydrogen peroxide.

In one aspect, the collagen hydrogel has a storage modulus of from about 200 Pa to about 650 Pa, or of from about 300 to about 400 Pa, or about 350 to about 400 Pa, or of about 200, 250, 300, 350, 400, 450, 500, 550, 600, or about 650 Pa, or a combination of any of the foregoing values, or a range encompassing any of the foregoing values. In still another aspect, the collagen hydrogel has a loss modulus of from about 60 to about 100 Pa, or about 75 to about 90 Pa, or about 80 to about 90 Pa, or of about 60, 65, 70, 75, 80, 85, 90, 95, or about 100 Pa, or a combination of any of the foregoing values, or a range encompassing any of the foregoing values.

### Method for Making a Collagen Hydrogel

Also disclosed herein is a method for preparing a collagen hydrogel, the method including at least the following steps:
(a) contacting a pre-gel solution of collagen with a solution of flavonoid in a solvent to create a first admixture;
(b) casting the first admixture in a container; and
(c) incubating the first admixture to produce the hydrogel.

In a further aspect, the pre-gel solution of collagen has a concentration of from about 0.5 to about 15 mg/mL, or of from about 1 to about 10 mg/mL, or of about 6 mg/mL. In another aspect, the solvent can be ethanol, dimethyl sulfoxide (DMSO), dimethylformamide (DMF), tetrahydrofuran (THF), propanol, lipid nanoparticles, methanol, ethyl acetate, acetonitrile, or any combination thereof. In still another aspect, the solvent can be ethanol. In one aspect, the first admixture can be incubated for about 5 minutes to about 1 hour, or for about 45 minutes. In some aspects, the admixture can be incubated for up to 16 hours. In another aspect, the first admixture can be incubated at a temperature of from about 4° C. to about 50° C., or at about 37° C. In other aspects, the first admixture can be incubated at a higher temperature. Without wishing to be bound by theory, the flavonoid crosslinker may provide additional thermostability to the collagen hydrogel. In any of these aspects, the first admixture is degassed prior to performing step (b) by a method such as, for example, vortexing, centrifugation, vacuum degassing, or any combination thereof. In one aspect, degassing can prevent the formation of bubbles in the collagen hydrogel or can remove bubbles introduced by mixing various components of the hydrogel. In any of these aspects, the method can be performed in a glass container, a plastic container, an inert metal container, a ceramic container, another nonporous material, or any combination thereof. In another aspect, the container can be a well plate, a centrifuge tube, a vial, a Petri dish, or another suitable container. In a further aspect, the container can withstand temperatures up to at least 121° C. at pressures of at least 15 psi so that the container can be sterilized by a method such as, for example, autoclaving prior to use.

### Cosmetics and Pharmaceutical Compositions

In one aspect, disclosed herein is a cosmetic or skincare composition including the disclosed collagen hydrogels and compositions. In another aspect, the cosmetic or skincare composition can be a moisturizer, body lotion, serum, wrinkle cream, eye cream, face mask, hand cream, sun protectant cream, sun protectant serum, or any combination thereof.

In another aspect, disclosed are pharmaceutical compositions including the disclosed collagen hydrogels and compositions. In a further aspect, disclosed herein is an article including the disclosed pharmaceutical compositions. In one aspect, the article can be selected from bandages, wound dressings, and the like. In another aspect, the wound dressing can be a gel, pad, sheet, or any combination thereof. In still another aspect, disclosed herein is a method for promoting or improving wound healing, the method including at least the step of applying a disclosed pharmaceutical composition or article to the wound. In another aspect, the wound can be a diabetic wound, a surgical incision, a burn, or another wound.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a flavonoid,” “a reactive oxygen species,” or “a collagen material,” include, but are not limited to, mixtures or combinations of two or more such flavonoids, reactive oxygen species, or collagen materials, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated+10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a flavonoid refers to an amount that is sufficient to achieve the desired improvement in the property modulated by the formulation component, e.g. achieving the desired level of elastic modulus and/or gel modulus. The specific level in terms of wt % in a composition required as an effective amount will depend upon a variety of factors including the amount and type of collagen, amount and type of flavonoid, and end use of the article made using the composition.

As used herein, the terms “optional” or “optionally” means that the subsequently described event or circumstance can or cannot occur, and that the description includes instances where said event or circumstance occurs and instances where it does not.

A “hydrogel” as used herein refers to a water-swollen, crosslinked polymeric structure containing either covalent bonds produced by the simple reaction of one or more co-monomers, physical cross-links from entanglements, association bonds such as hydrogen bonds or strong van der Waals interactions between chains, or crystallites bridging together two or more macromolecular chains. In one aspect, in the disclosed hydrogels, luteolin provides at least a portion of the covalent crosslinks.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.
Aspect 1. A composition comprising a collagen hydrogel and a flavonoid, wherein at least a portion of the collagen hydrogel is crosslinked by the flavonoid.
Aspect 2. The composition of aspect 1, wherein the flavonoid comprises a flavonol, flavone, anthocyanidin, isoflavonoid, neoflavonoid, an anthoxanthin, a derivative thereof, or any combination thereof.
Aspect 3. The composition of aspect 2, wherein the flavonol comprises catechin, gallocatechin, catechin 3-gallate, gallocatechin 3-gallate, epicatechin, epigallocatechin, epicatechin 3-gallate, epigallocatechin 3-gallate, leucoanthocyanidin, a derivative thereof, or any combination thereof.
Aspect 4. The composition of aspect 2, wherein the anthocyanidin comprises anthocyanidin, cyanidin, delphinidin, malvidin, pelargonidin, peonidin, petunidin, a derivative thereof, or any combination thereof.
Aspect 5. The composition of aspect 2, wherein the isoflavonoid comprises genistein, daidzein, glycitein, equol, lonchocarpan, laxiflorane, a derivative thereof, or any combination thereof.
Aspect 6. The composition of aspect 2, wherein the neoflavonoid comprises calophylloid, dalbergichromene, coutareagenin, dalbergin, nivetin, a derivative thereof, or any combination thereof.
Aspect 7. The composition of aspect 2, wherein the anthoxanthin comprises quercetin, betaxanthin, canthaxanthin, a derivative thereof, or any combination thereof.
Aspect 8. The composition of aspect 2, wherein the flavone comprises primuletin, chrysin, tetochrysin, primetin, apigenin, acacetin, genkwanin, echoidinin, baicalein, oroxylin A, negletein, norwogonin, wogonin, liquiritigenin, naringenin, geraldone, tithonine, luteolin, 6-hydroxyluteolin, chrysoeriol, diosmetin, pillion, velutin, norartocarpetin, artocarpetin, scutellarein, hispidulin, sorbifolin, pectolinarigenin, cirsimaritin, mikanin, isoscutellarein, zapotinin, zapotin, cerrosillin, alnetin, tricetin, tricin, corymbosin, nepetin, pedalitin, nodifloretin, jaceosidin, cirsiliol, eupatorine, sinensetin, hypolaetin, onopordin, wightin, nevadensin, xanthomicrol, tangeretin, serpyllin, sudachitin, acerosin, hymenoxin, gardenin D, nobiletin, scaposin, a derivative thereof, or any combination thereof.

9 Aspect. The composition of aspect 2, wherein the flavone is luteolin.
Aspect 10. The composition of any one of aspects 1-9, wherein the collagen hydrogel comprises type I collagen, type II collagen, type III collagen, type IV collagen, type V collagen, type VI collagen, type VII collagen, type VIII collagen, type IX collagen, type XI collagen, type XXI collagen, or any combination thereof.
Aspect 11. The composition of any one of aspects 1-10, wherein the collagen hydrogel comprises bovine collagen, porcine collagen, chicken collagen, turkey collagen, egg collagen, fish collagen, recombinant collagen produced by bacterial fermentation, extracellular matrix (ECM)-derived collagen, or any combination thereof.
Aspect 12. The composition of aspect 11, wherein the recombinant collagen produced by bacterial fermentation comprises bovine collagen, porcine collagen, chicken collagen, turkey collagen, egg collagen, fish collagen, human collagen, or any combination thereof.
Aspect 13. The composition of aspect 10, wherein the collagen comprises a combination of type I collagen and type III collagen.
Aspect 14. The composition of any one of aspects 1-13, wherein the composition is cytocompatible.
Aspect 15. The composition of any one of aspects 1-14, wherein the composition protects cells from oxidative damage.
Aspect 16. The composition of any one of aspects 1-15, wherein the composition comprises from about 5 μM to about 400 μM of the flavonoid.
Aspect 17. The composition of any one of aspects 1-15, wherein the composition comprises from about 40 μM to about 120 μM of the flavonoid.
Aspect 18. The composition of any one of aspects 1-17, wherein the collagen hydrogel has an average pore size of from about 0.5 μm to about 10 μm.
Aspect 19. The composition of any one of aspects 1-17, wherein the collagen hydrogel has an average pore size of from about 0.5 μm to about 3 μm.
Aspect 20. The composition of any one of aspects 1-19, wherein the collagen hydrogel has a Trolox equivalent antioxidant capacity (TEAC) value of at least about 2 mM/mg.
Aspect 21. The composition of any one of aspects 1-20, wherein the collagen hydrogel retains at least about 45% metabolic activity relative to an untreated control after exposure to 300 μM hydrogen peroxide.
Aspect 22. The composition of any one of aspects 1-21, wherein the collagen hydrogel has a storage modulus of from about 200 Pa to about 650 Pa.
Aspect 23. The composition of any one of aspects 1-22, wherein the collagen hydrogel has a loss modulus of from about 60 Pa to about 100 Pa.
Aspect 24. A method for preparing a collagen hydrogel, the method comprising:
   (a) contacting a pre-gel solution of collagen with a solution of flavonoid in a solvent to create a first admixture;
   (b) casting the first admixture in a container; and
   (c) incubating the first admixture to produce the hydrogel.
Aspect 25. The method of aspect 24, wherein the pre-gel solution of collagen has a concentration of from about 0.5 to about 15 mg/mL.
Aspect 26. The method of aspect 24, wherein the pre-gel solution of collagen has a concentration of about 6 mg/mL.
Aspect 27. The method of any one of aspects 24-26, wherein the solvent comprises ethanol, dimethyl sulfoxide (DMSO), dimethylformamide (DMF), tetrahydrofuran (THF), propanol, lipid nanoparticles, methanol, ethyl acetate, acetonitrile, or any combination thereof.
Aspect 28. The method of any one of aspects 24-27, wherein the container comprises a glass container, a plastic container, an inert metal container, a ceramic container, another nonporous material, or any combination thereof.
Aspect 29. The method of any one of aspects 24-28, wherein the container comprises a well plate, a centrifuge tube, a vial, or a Petri dish.
Aspect 30. The method of any one of aspects 24-29, wherein the container can withstand temperatures up to at least 121° C. at pressures of at least 15 psi.
Aspect 31. The method of any one of aspects 24-30, wherein the first admixture is incubated for up to 16 hours.
Aspect 32. The method of any one of aspects 24-30, wherein the first admixture is incubated for from about 5 min to about 1 hour.
Aspect 33. The method of any one of aspects 24-30, wherein the first admixture is incubated for about 45 min.
Aspect 34. The method of any one of aspects 24-33, wherein the first admixture is incubated at a temperature of from about 5° C. to about 50° C.
Aspect 35. The method of any one of aspects 24-33, wherein the first admixture is incubated at about 37° C.
Aspect 36. The method of any one of aspects 24-35, wherein the first admixture is degassed prior to performing step (b).
Aspect 37. A cosmetic or skincare composition comprising the composition of any one of aspects 1-23.
Aspect 38. The cosmetic or skincare composition of aspect 37, wherein the cosmetic or skincare composition comprises a moisturizer, body lotion, serum, wrinkle cream, eye cream, face mask, hand cream, a sun protectant cream, a sun protectant serum, or any combination thereof.
Aspect 39. A pharmaceutical composition comprising the composition of any one of aspects 1-23.
Aspect 40. An article comprising the pharmaceutical composition of aspect 39.
Aspect 41. The article of aspect 40, wherein the article comprises a bandage or a wound dressing.
Aspect 42. The article of aspect 41, wherein the wound dressing comprises a gel, pad, sheet, or any combination thereof.
Aspect 43. A method for promoting or enhancing wound healing, the method comprising applying a pharmaceutical composition according to aspect 39 or an article according to any one of aspects 40-42 to a wound.
Aspect 44. The method of aspect 43, wherein the wound comprises a diabetic wound, a burn, or a surgical incision.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Methods and Materials

### Materials

All materials were used without further purification. FIBRICOL® (available from Advanced BioMatrix, Inc., San Diego, CA), Bovine Collagen Solution (10.4 mg/mL) and Sodium Hydroxide solution suitable for cell culture was purchased from Millipore-Sigma. Quality Biological™ 10× PBS, Quality Biological™ 1× PBS, Quality Biological™ DMEM with Glutamine XL, Sterile and Nuclease-free Water (all available from Quality Biological, Inc., Gaithersberg, MD), KOPTEC Absolute Ethanol (anhydrous, 200 Proof), Hydrogen peroxide 3% (w/w) in aqueous solution stabilized, and Penicillin-Streptavidin solution were purchased from VWR. Luteolin 99.4% was purchased from Santa Cruz Biotechnology Inc. Normal Human Adult Fibroblasts cryopreserved ampule (CC-2511) containing≥500.000 cells from a 26 year old female donor and FGM™-2 Fibroblast Growth Medium-2 BulletKit™ (available from Lonza Walkersville, Inc., Walkersville, MD) were purchased from Lonza Bioscience. Fetal Bovine Serum, Heat Inactivated, Trypsin-EDTA 0.25%, Genclone tissue culture treated well plates, and Genclone tissue culture treated flasks were purchased from Genesee Scientific. Invitrogen™ LIVE/DEAD™ Viability/Cytotoxicity Kit, for mammalian cells (available from Molecular Probes, Inc., Eugene, OR), Abcam MTT Assay Kit (Cell Proliferation), and Cayman Chemical Antioxidant Assay Kit were purchased from Fisher Scientific. 2.8 mm ceramic beads were purchased from Omni International.

### Hydrogel Preparation and Self Assembly

Collagen hydrogels were prepared according to the manufacturer's instructions. Briefly, FIBRICOL® collagen solution (0.6 mL, 10.4 mg/mL) was salt and pH adjusted with 10× PBS and 0.1M NaOH on ice to create a pre-gel solution of 6 mg/mL collagen. Luteolin stock solutions in absolute ethanol were freshly prepared, and 12 μL from each stock solution was added to 1 mL aliquots of pre-gel solution to create the respective luteolin-collagen pre-gel formulations at 0,0 with vehicle (12 μL ethanol) EtOH, 40, 80, and 120 μM luteolin. Gels were vortexed and briefly spun down to remove bubbles. Gels were cast in tissue culture-treated 96 well plates and incubated for 45 min. at 37° C. to afford fully self-assembled hydrogels.

### Physical Hydrogel Characterization

Rheological Measurements. All rheological measurements were conducted using a Discovery Hybrid Rheometer-3 (TA Instruments, New Castle, DE). A 40 mm sandblasted parallel plate configuration was used to prevent walls slippage. A sample gap of 200 μm was maintained for all experiments, and a solvent trap used to prevent evaporation. After loading the pre-gel sample prepared using the above-mentioned procedure into the rheometer geometry maintained at 4° C., the samples were conditioned by subjecting them to a dynamic preshear of 0.1 rad/s for 10 seconds and then allowing them to stabilize for 2 minutes. The evolutions of the elastic (G′) and viscous (G″) moduli were then monitored over time at a constant frequency of 1 rad/s, as the temperature of sample rapidly increased from 4 to 37° C. The time sweep experiment was followed with measuring the elastic (G′) and viscous (G″) modulus as a function of frequency. All experiments were conducted at 1% strain, well within the linear viscoelastic (LVE) regime, and carried out in triplicate to ensure reproducibility.

Turbidity Measurements. Hydrogels were prepared as described above and 50 μL of gel solution was cast into 96 well plates and kept on ice until analysis. Plates were inserted into a Tecan Infinite M PLEX plate reader (Männedorf, Switzerland), and samples were read at an absorbance of 405 nm every 1 minute for a total of 90 minutes at 37° C. Each experiment contained 5 replicates of each luteolin-collagen gel, and the experiment was run in triplicate. Data was analyzed according to Equation 1.

$$Normalized Absorbance 405 nm = \frac{( Ax - A min )}{( A max - A min )} \qquad ( 1 )$$

where Ax=experimental measurement, Amin=minimum absorbance, and Amax=maximum absorbance

Cryo-SEM. Gels were prepared as described above and cast directly into the Cryo-SEM sample holder and incubated at 37° C. for 1 hour to induce gelation. Cryo-SEM analyses were conducted using a JEOL JSM-7600 FE SEM (JEOL USA, Peabody, MA) outfitted with Alto-2500 (Gatan, Warrendale, PA). Samples were plunge-frozen in liquid nitrogen slush, transferred under vacuum to the Alto-2500 preparation chamber and cryo fractured. The fractured sample was etched for 5 min at −89° C. under 4×10-6 mbar vacuum to reveal the microstructure. Samples were allowed to cool down to −135° C. An in-situ cold magnetron coater was used to make a 5 nm thickness Au/Pd coating on etched samples. The SEM images were taken using 5 keV energy at 11 mm working distance under cryo-temperature. Pore sizes were measured after thresholding with FIJI image processing software. At least 5 entire images were analyzed per sample.

Antioxidant Activity. Antioxidant activity assays (Cayman Chemical, Ann Arbor, MI) were performed according to the manufacturer's instructions using 10 μL aliquots of 0, 40, 80, 120 μM luteolin-collagen pre-gel solutions added to the Trolox reagents in a 96 well plate. Reactions in each well were initiated with 40 μL of 44 μM H₂O₂ and agitated for 5 minutes prior to absorbance readings at 750 nm using a Tecan Infinite M PLEX plate reader (Männedorf, Switzerland). Data was analyzed as previously described in the literature and expressed as mM Trolox equivalents per mg of the gel formulation sample. Equation 2. Six separate experiments were conducted with at least three technical replicates per experiment per sample. One-Way ANOVA analysis was performed to determine statistical significance.

$$Trolox Equivalents ( \frac{mM}{mg} ) = \frac{[ \frac{( Sample average absorbance ) - ( y intercept )}{S lope} ]}{mg sample} \qquad ( 2 )$$

### Cell Culture and Cytocompatibility

Cell Culture. Human Dermal Fibroblasts (NADFbs) were purchased from Lonza Biosciences and cultured according to the manufacturer's instructions. Briefly, Cells were seeded at a density of 4000 cells/cm2 into 75 cm2 tissue culture treated flasks supplemented with FGM™-2 Fibroblast Growth Medium-2. Cells were maintained at 37° C. with 5% CO2 and 20% oxygen, and maintenance media was changed every 2 days. Cells were passaged once they reached 80% confluency using 0.25% Trypsin EDTA for 5 minutes and then neutralized with maintenance media of DMEM containing GLUTAMINE XL, 10% fetal bovine serum and 1% penicillin/streptavidin and seeded into tissue culture treated flasks. Cells were subsequently maintained in this media. Passages 4-11 were used in cell studies, as specified in the subsequent sections.

LIVE/DEAD. NADFbs (passages 4,6) were seeded at a density of 5×10⁴ cells/well on top of 400 μL amount of gel (0-120 M Luteolin) cast in 12 well plates for 2D cell culture. As a control, cells were seeded as described above without gels directly onto 12 well plates. 1 ml of culture media was added to each well and cells were cultured for 48 hours at 37° C. After 48 hours, wells were washed 3×1 mL 1×PBS and then incubated with LIVE/DEAD red/green fluorescence intensity staining solution (ThermoFisher Scientific, Waltham, MA) for 20 minutes at room temperature protected from light. The staining solution was discarded and replaced with media. The samples were imaged using an Olympus fluorescence microscope at 20× magnification and counted using FIJI image processing software. Experiments were conducted in duplicate with n=2 sample replicates and n=4 technical replicates, 16 replicates total per sample. One-Way ANOVA analysis was performed to determine statistical significance.

MTT. NADFbs (passages 4,6,8) were seeded at a density of 9×10³ on top of 50 μL of gel (0-120 μM Luteolin) cast in 96 well plates for 2D cell culture or at a density of 9×10³ inside of 50 μL pre-gel solution (0-120 μM Luteolin)/well and cast in 96 well plates for 45 mins at 37° C. for 3D cell culture. As a control, cells were seeded as described above without gels directly onto 96 well plates. 200 μL media was added to each well and cells were cultured for 48 hours. After 48 hours, wells were washed 3×100 μL 1×PBS and then incubated with 50 μL 3-(4,5-dimethylthiazol-2-yl)-2,5-diphenyl tetrazolium bromide (MTT) reagent solution and 50 μL of serum and phenol-red free media for 3 hours at 37° C. according to the manufacturer's instructions. Gels and solution were removed from the plate, homogenized using a Fisherbrand™ Bead Mill 24 Homogenizer (Fisher Scientific, Waltham, MA) with 150 UL MTT solvent solution, and transferred carefully to a new 96 well plate. Solutions were read at 595 nm using a Tecan Infinite M PLEX plate reader (Männedorf, Switzerland). Experiments were conducted in triplicate with n=3 replicates/sample/experiment. Results were calculated according to Equation 3 and were plotted using the mean and standard deviation of each sample. One-Way ANOVA analysis was performed to determine statistical significance.

$$Percent Metabolic Activity = [ \frac{Lut}{C} ] \times 100 \qquad ( 3 )$$

where C=the average absorbance reading taken from 0 μM samples at 590 nm after background subtraction of a negative control (no cells) and Lut=average absorbance reading taken from each Luteolin hydrogel sample (40-120 μM) at 590 nm after background subtraction of a negative control (no cells).
Cellular Protection from Oxidative Damage

MTT. NADFbs (passages 8,9,11) were seeded at a density of 2×10⁴ cell/well inside of 50 μL pre-gel solution (0-120 μM Luteolin)/well and cast in 96 well plates for 45 mins at 37° C. for 3D cell culture. 100 UL of DMEM with 5% FBS and phenol-red free cell culture media was added to each well and cells were cultured for 1 hour. Following initial culture, 100 μL of 600 μM H₂O₂ in identical media was added to the cell-laden hydrogels to yield a 300 μM H₂O₂ solution. This challenge was performed for 2 hours at 37° C. Cells were then washed 3×100 μL 1×PBS, and the MTT assay was performed as described previously. Experiments were conducted in triplicate with n=3 replicates/sample/experiment. Results were calculated according to Equation 3 and were plotted using the mean and standard deviation of each sample. One-Way ANOVA analysis was performed to determine statistical significance.

### Statistical Analysis

GraphPad PRISM 9.0 software was used to plot data and perform statistical analyses. Unless written otherwise, all experiments were performed at least three independent times. Statistical significance was calculated using a One-Way ANOVA with a post-hoc Tukey's Test and defined as a value of p<0.05.

### Example 2: Results

### Hydrogel Preparation

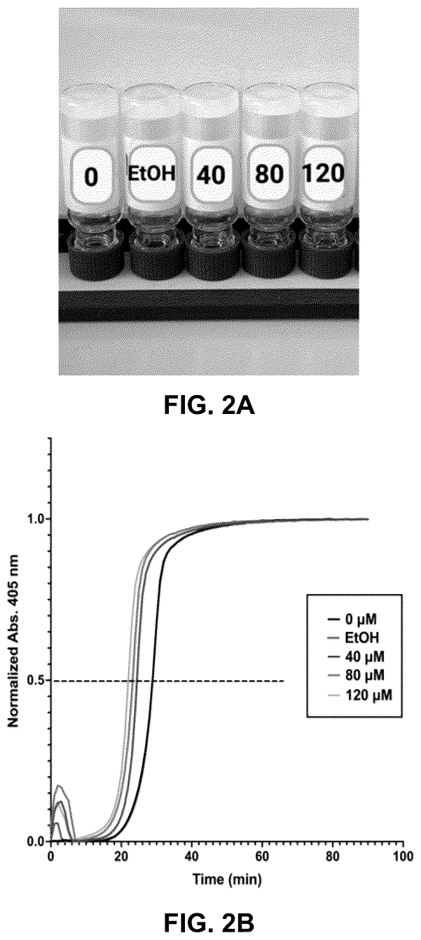

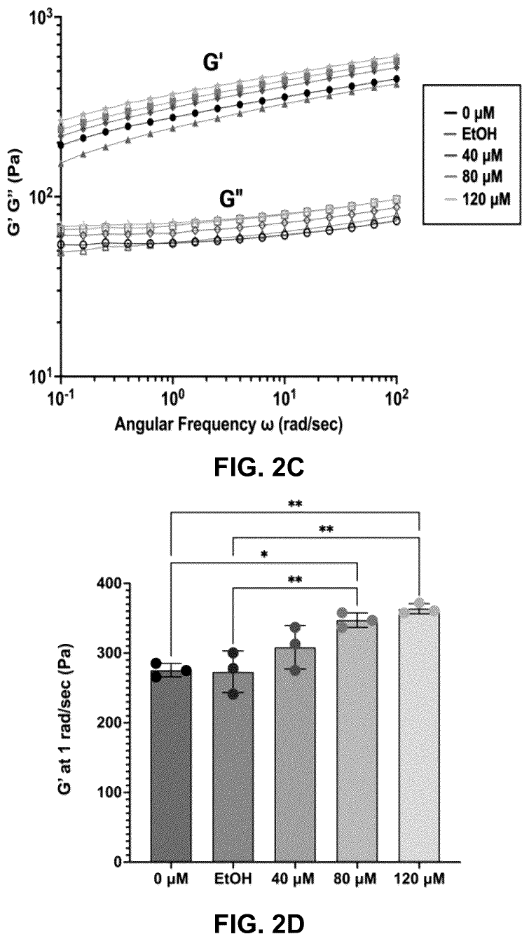

Collagen hydrogels were prepared according to a modified protocol to accommodate the introduction of luteolin to the formulation. Luteolin stock in ethanol (12 μL) was added without complication to 0.6 mL of bovine collagen solution after salt and pH adjustment. Gels were cast at 37° C. for 45 mins. All luteolin-collagen formulations produced robust hydrogels after 45 minutes as observed from an inversion test conducted at room temperature (FIG. 2A).

### Rheology and Kinetics

Dynamic rheological experiments were conducted to observe the sol-gel transition of the system, which is important because the material needs to gel rapidly upon exposure to body temperature (37° C.) at the site of any defect. In the time dependent progression of elastic (G′) and viscous (G″) moduli for collagen hydrogels with 0 μM, EtOH, and 120 μM luteolin, the samples are initially a solution with G″>G′, but as the temperature reaches 37° C., both moduli begin to increase, with a crossover occurring at around 2-3 minutes. This crossover point is considered to be the approximate gelation time for the hydrogels. After the cross-over point, both moduli continue to grow with G′ exceeding G″ and eventually asymptoting to a steady value. The frequency sweep results at 37° C. (FIG. 2C) for all the samples (0-120 μM), show G′>G″ and both the moduli are independent of frequency over the entire range (0.1-100 rad), suggestive of a gelled network. FIG. 2D illustrates the values of elastic modulus (G′) for all samples at 1 rad/s. The gel modulus values for collagen-only sample (0 μM) and the collagen solution containing the ethanol carrier (EtOH) are 285.45+5.60 Pa and 273.085+17.27 Pa, respectively. At a frequency of 1 rad/s, the elastic modulus value increases with increasing concentrations of luteolin and reaches its peak value of 363.67+4.25 Pa for a collagen hydrogel containing 120 μM luteolin.

### Macromolecular Structure

Turbidity measurements, a metric for optically measuring the progress of hydrogel self-assembly, were conducted by monitoring the gels at increasing concentrations of luteolin at 37° C. over a period of 90 minutes at 405 nm (FIG. 2B). Sigmoidal behavior was observed for all gelation curves. Table 1 summarizes the measured amount of time needed to reach 50% of the maximum turbidity (t/2). A positive correlation was observed between the concentration of luteolin and decreasing half time, and all luteolin-collagen samples displayed strong statistical significance (p<0.0001) when comparing turbidity t/2 to those of 0 μM and collagen solution incorporating only vehicle, EtOH. A significant difference of 6.91 min. (p<0.0001) was measured between the average 0 μM time (28.75 min.+0.50) and the 120 μM time (21.84 min+0.58). EtOH showed no significant deviation in gelation behavior compared to 0 M.

TABLE 1
Time to Reach 50% Turbidity
(mean ± standard deviation)

| Sample | Time (min) |
|---|---|
| 0 μM | 28.75 ± 0.50 |
| EtOH | 29.00 ± 0.50 |
| 40 μM^{a,b} | 24.63 ± 0.63 |
| 80 μM^{a,b} | 22.84 ± 0.58 |
| 120 μM^{a,b} | 21.84 ± 0.58 |

^{a}Statistically different form control 0 μM, p < 0.0001
^{b}Statistically different form control EtOH, p < 0.0001

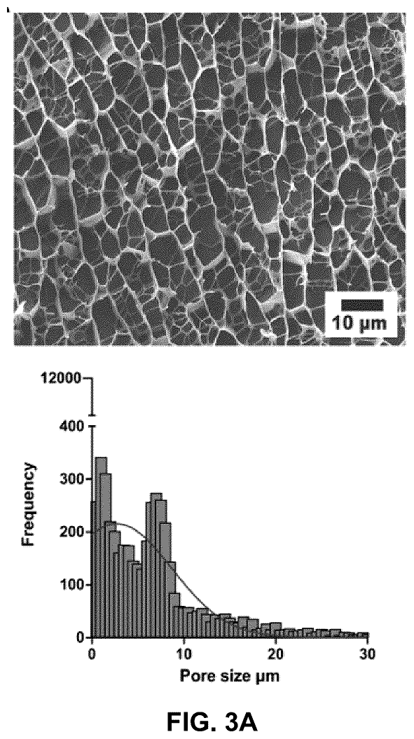

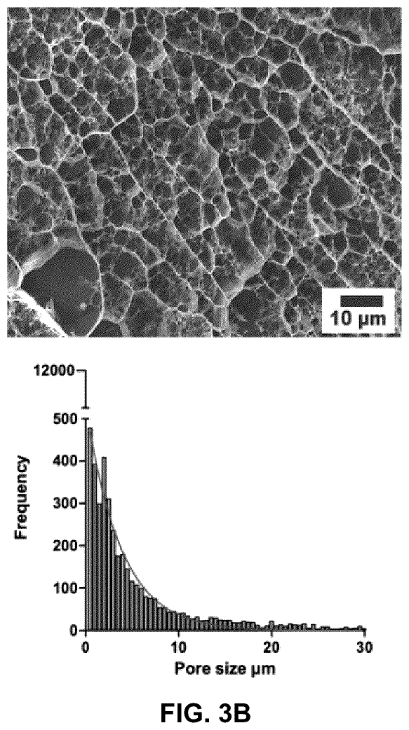

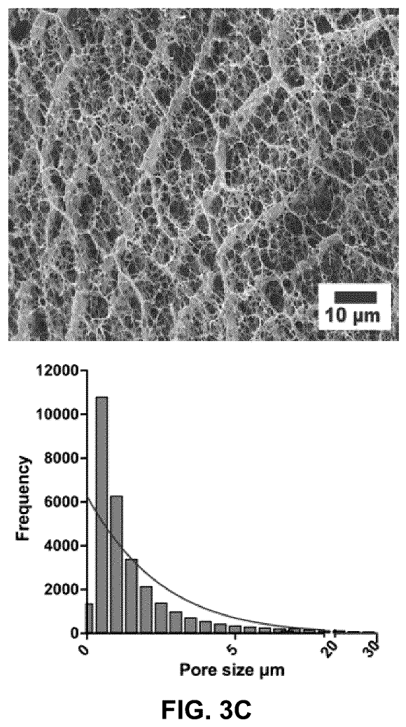

The macromolecular structures of the hydrogels were imaged by using cryo-scanning electron microscopy (cryoSEM). Gels were prepared directly into SEM sample holders to minimize structural perturbations and incubated at 37° C. for 1 hour to complete gelation. Representative SEM images are shown in FIGS. 3A-3C (top). Protein crosslinks appear in images of 80 μM (FIG. 3B) and increase in a concentration-dependent manner throughout images of 120 μM gels (FIG. 3C). Increased crosslinks resulted in reduction of the pore size. Median pore sizes were measured to be 6.31 μm+10.69, 3.51 μm+16.92, and 0.999 μm+4.88 for 0 μM, 80 μM, and 120 μM gels, respectively. Distribution of pore sizes also varied between the scaffolds: 0 μM displayed bimodal distribution with clustering around 0.5-2.0 μm and 6.0-8.5 μm and a tapering measurement of larger pore sizes up to about 20.0 μm. With luteolin present in the 80 μM scaffold, the distribution changed to a unimodal presentation, with major clustering under 3.0 μm, yet larger pore sizes up to 17.0 μm were prevalent. As the concentration of luteolin increased to 120 μM, the distribution continued to be unimodal with clustering between 0.5-2.0 μm, and the majority of the measured pore size population remained under 3.0 μm (FIGS. 3A-3C (bottom)).

### Cytocompatibility

Cytocompatibility was evaluated as the collection of results from LIVE/DEAD and MTT assays using cells in 2D and 3D culture in dose-response experiments with increasing concentrations of luteolin within the hydrogels. Briefly, NADFb (passages 4,6) were cultured in either 2D or 3D with the hydrogels (0-120 M) for 48 hours prior to LIVE/DEAD staining and MTT analysis.

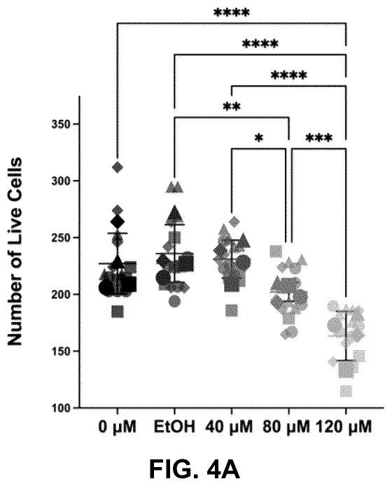

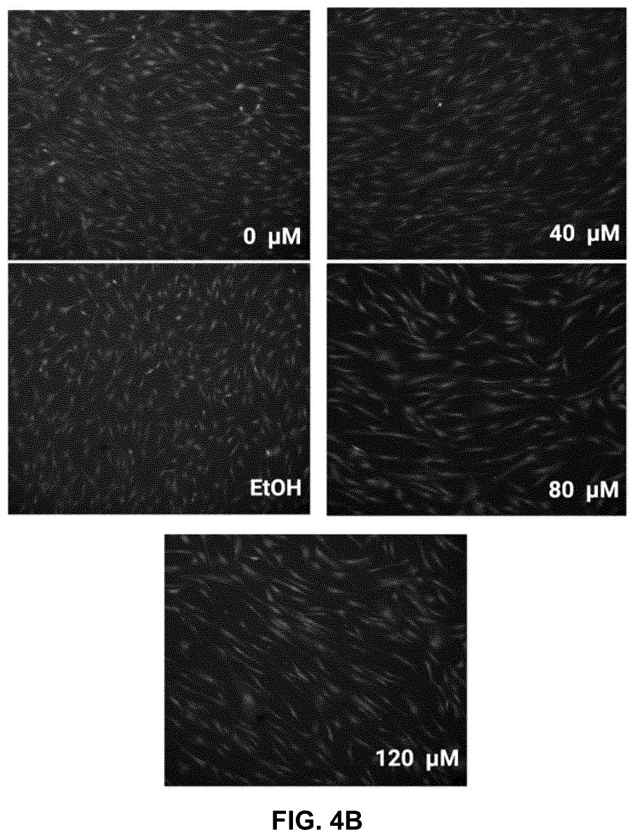

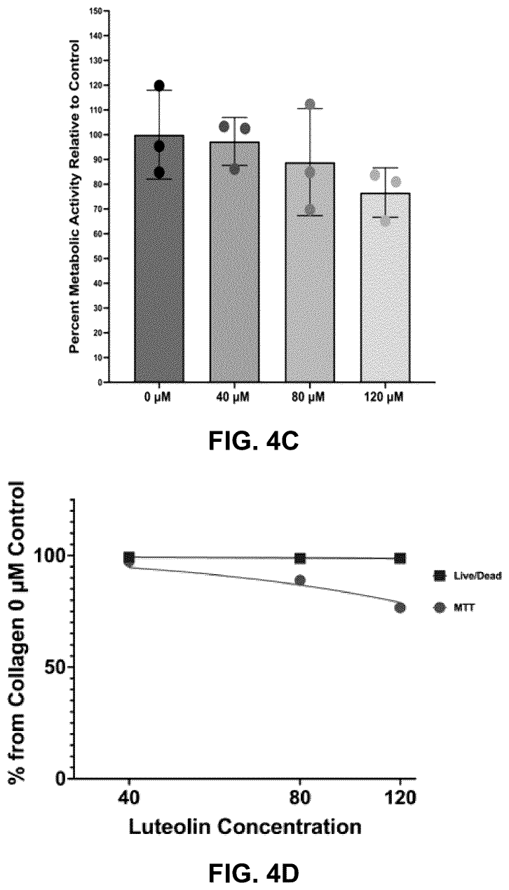

Representative LIVE/DEAD images are shown in FIG. 4B and display high numbers of cultured healthy cells cultured in 2D upon the gels and a modest drop in live cell counts with increasing concentrations of luteolin: 0 μM, 227+31.26; EtOH, 236+31.21; 40 μM, 231+19.50; 80 μM, 202+22.63; 120 μM, 163+22.76 (FIG. 4A). Yet, cell viabilities of 99.5-98.75% were observed regardless of hydrogel formulation (FIG. 4D).

MTT assays for 2D and 3D cell cultures showed a trend in metabolic activity that follows the live cell counts reported from LIVE/DEAD experiments (FIG. 4C). Gradual decreases in metabolic activity were observed for cells as the luteolin content of the gels was increased: 0 μM, 100%+17.96; 40 μM, 97.26%+9.72; 80 μM, 88.92%+21.62; 120 μM, 76.65%+10.00. However, one-way ANOVA analysis did not find these differences in metabolic activity to be statistically significant.

### Antioxidant Functionality of Flavonoid Hydrogels

To assess the gels' efficacy as an antioxidant material, Trolox assays were performed with acellular hydrogels, as expressed in Trolox equivalents per mg of material. Trolox is a water-soluble vitamin E analog, and this assay has been considered the gold star in assessment of a substance's ability to scavenge the radical 2,2′-azinobis-(3-ethylbenzothiazoline-6-sulphonic acid) cations through colorimetric analysis.

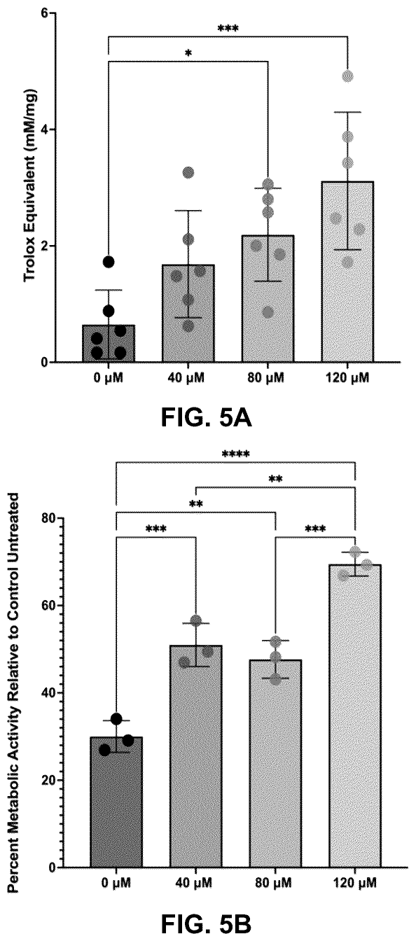

As previously reported, luteolin is a naturally occurring antioxidant. Thus, it is unsurprising that increasing concentrations of luteolin would afford higher Trolox equivalent values in the presence of hydrogen peroxide (FIG. 5A). While collagen-only hydrogel, 0 μM, was calculated to have 0.65+0.59 mM/mg equivalent radical scavenger activity to Trolox, the luteolin formulations resulted in the following values: 40 μM, 1.69±0.92; 80 μM, 2.19±0.80; and 120 μM, 3.12±1.18 mM/mg luteolin-collagen sample relative to Trolox. Statistical significance was calculated between hydrogels of 0 μM and 80 μM (p=0.0349), and 120 μM luteolin gels considerably enhanced the antioxidant effect compared to 0 μM (p=0.0007). 120 μM demonstrated a 4.8-fold increase of antioxidant activity relative to the 0 μM formulation.

The capacity for luteolin-collagen hydrogels to exert biologically protective effects against ROS in a model environment was conducted as a measure of metabolic activity following incubation with 300 μM hydrogen peroxide for 2 hours. To ensure minimal interference from nutrient content in media, 3D cultures of NADF were challenged with hydrogen peroxide in a reduced concentration of serum and phenol red-free media for the duration of the ROS incubation. Experiments were repeated in triplicate with 3 replicates per sample per experiment. Results were interpreted as the percent metabolic activity of cells normalized to control untreated 0 μM hydrogels.

Across all luteolin-collagen hydrogel formulations, a biologically protective effect could be calculated as a conservation of metabolic function after ROS challenge (FIG. 5B). Greater preservation of metabolic activity was observed concomitantly with luteolin content increase: 0 μM, 30.02%±3.64; 40 μM, 50.97%±4.96; 80 μM, 47.67%±4.32; 120 μM, 69.47%±2.73. Among the different luteolin formulations, statistically significant differences in metabolic activity were determined between 0 μM and the luteolin formulations: 40 μM p=0.0009; 80 μM p=0.0029; 120 μM p<0.0001. Notably, high significance was measured when comparing 120 μM to lower concentration hydrogels: 40 μM p=0.0021; 80 μM p=0.0007. No statistical significance was determined between 40 μM and 80 μM. Hydrogels containing 120 μM provide a 2.3-fold enhancement in protection from oxidative-induced metabolic impairment when compared to hydrogels formulated without luteolin.

### Example 3: Discussion and Conclusion

### DISCUSSION

The goal of this study was to determine the influence of luteolin on collagen hydrogel structure and self-assembly, biocompatibility, and antioxidant efficiency through physical characterization, in vitro biological evaluation, and antioxidant challenges. It was hypothesized that the combined use of a well-known antioxidant (Luteolin), a hydrogel material possessing desirable wound healing and tissue engineering characteristics (collagen), and a supplemental dose of viable cells (NADFbs) could yield more potent antioxidant effects in vitro, which could potentially offer protective benefits in future in vivo studies. This hydrogel combination could potentially impart momentum behind the healing progression in chronic and complex wounds through neutralizing elevated ROS species and protecting payload cells and the wound site from oxidative damage. Luteolin concentrations were selected based on previous in vitro and in vivo experimentation using a range of millimolar to micromolar concentrations with low toxicity for skin therapeutic purposes. Luteolin stock solutions dissolved in ethanol were highly compatible with salt and pH adjusted collagen solutions and incorporated readily without further modification of the collagen formulation or hydrogel protocol. Regardless of the concentration of luteolin added, all gels assembled into robust, self-standing hydrogels within 45 minutes of incubation at 37° C., as determined by an inversion test.

Rheological measurements provide insight into the kinetics of gelation and the ultimate strength of the hydrogels. Based on the time-dependent results, it appears that the inclusion of ethanol or luteolin does not have a substantial impact on the gelation time of the collagen hydrogels. However, examining beyond the gel point the hydrogel was found containing only ethanol, EtOH, to exhibit slower kinetics in reaching the final modulus in comparison to the collagen hydrogel alone. At physiological temperature, the presence of ethanol, a less polar substance than water, in collagen solution can disrupt the water mediated inter-strand hydrogen bonding resulting in a slower gelling process. When luteolin dissolved in ethanol is combined into the collagen sample, the gelation kinetics regain its momentum, indicating the possible formation of additional bonds between the collagen fibrils and luteolin. This was further substantiated by studying the frequency-dependent behavior of the samples at 37° C. The results (FIGS. 2A-2D) indicate that all samples form a sample-spanning gel network, and their gel modulus increases with an increase in the concentration of luteolin.36 The formation of stronger hydrogels could potentially be attributed to the formation of non-covalent interactions such as hydrophobic interactions and hydrogen bonding. Song et al. found that integration of luteolin in poly(vinyl alcohol) forms self-healing hydrogel with strong mechanical properties due to the formation of non-covalent interactions. Taken together, these results indicate that the addition of luteolin to collagen solution does not alter the gelation time or kinetics, but enhances the overall strength of the hydrogel.

Traditionally, turbidity measurements are used to establish the dynamics of the gelation process. Although rheological results do not demonstrate any discernable change in gelation kinetics with increasing concentration of luteolin, the turbidity assessment at 405 nm shows an increase in gelation rate with a positive correlation between luteolin concentration and decrease in gelation time. Considering the significant departure in macromolecular structure: the degree of crosslinking and decline in pore size with the addition of luteolin, the obscuring of light and the interference of its penetration through the collagen-luteolin hydrogel matrix once gelation has commenced appear to be the responsible for the linear increase in absorbance. Thus, in this instance, the turbidity measurements do not necessarily reflect kinetics of gelation, only that the differences in scaffold architecture are influencing the absorbance measurements. Despite this, this data reinforces that the disclosed methodology for cryoSEM analysis was appropriate and preserved the structural integrity of the hydrogel formed under normal conditions. Furthermore, by examining the shape of the graphs, it can be concluded that the sigmoidal curve associated with each luteolin hydrogel is indicative that the inclusion of luteolin within the hydrogel formulation does not disrupt the cooperative hydrogel self-assembly.

CryoSEM images depicted luteolin's influence on collagen hydrogel ultrastructure in a concentration-dependent manner and revealed a substantially different scaffold architecture compared to traditional collagen hydrogels. An increase in the fine interconnected network of crosslinks between larger collagen fibrils caused 120 M hydrogels to resemble a dense mesh under cryoSEM magnification. As a result, the median pore size was reduced to 0.99 μm with a unimodal distribution of pore size, about 6-fold smaller than a typical collagen hydrogel. Gels without luteolin (0 μM gels) revealed a bimodal distribution of pore size, displaying large populations of pore sizes 6-8 μm, but also the occurrence of smaller sized pores from smaller crosslinks as seen in 120 μM. Hydrogels containing 80 μM of luteolin resembled a midway point between the 0 μM and 120 μM gels relative to the occurrence in fine crosslinking and the reduction in pore sizes. The median pore size within 80 μM gels was measured to be 3.5 μm, 3 times larger than those of 120 μM gels, but also larger pore sizes were observed similar to those in 0 μM. Pore size is an important characteristic of wound dressings, with sufficient pore sizes and porosities of the scaffold allowing exchange of nutrients, passage for cell migration to the site, and room for proliferation. However, the disclosed proposed therapy, delivering a large payload of viable fibroblasts as the collagen hydrogel naturally degrades in vivo, circumvents the need for larger pores to accommodate the migration and proliferation of endogenous fibroblasts in the wound bed. Taken together with the rheology data, it is hypothesized that the stronger luteolin-collagen gel formulations can act in a similar manner as typical collagen hydrogels to protect the wound against dehydration, detrimental mechanical forces, and debris and biological pathogens, while maintaining embedded cell health and viability.

Physical characterizations of gels containing only vehicle, ethanol, without luteolin were conducted to ensure the inactive formula ingredients did not influence the gel's physical character. Despite being an established crosslinking agent, the inclusion of ethanol in the formulation did not enhance the kinetics of gelation, produce additional protein contacts within the gel matrix, or enhance the gel's strength, as witnessed by rheological, turbidity, and cryoSEM analysis (FIGS. 2A-3C).

Although the individual ingredients of the disclosed luteolin-collagen hydrogel formulations have undergone cytocompatibility tests in vitro, once combined into one hydrogel material, the cellular compatibility of these materials was confirmed through fluorescent staining and mitochondrial activity. LIVE/DEAD confirmed high numbers of viable cells with a modest, dose-dependent decrease in cell count. MTT analysis calculated slight dose-dependent decreases in metabolic activity as well, 97-77% depending on concentration, but results were not determined to be statistically significant. Despite this trend, percent viability of cells was greater than 98% across all luteolin-gel formulations (FIG. 4D), suggesting a proliferative mechanism may be diminished, and luteolin does not induce cellular toxicity. Such behavior was also observed when tannic acid microparticles were included in collagen hydrogel formulations. Additional studies are necessary to confirm the exact explanation of the cellular behavior in increasing luteolin concentrations. Concentrations greater than 120 μM led to significantly decreased cell count and mitochondrial function and were not progressed further in this study (Data Not Included).

Luteolin belongs to a class of molecules called flavonoids, and contains multiple aromatic systems and other radical neutralizing features capable of managing unpaired electrons. Due to its established chemical behavior as an antioxidant, it was hypothesized that collagen hydrogel formulations incorporating luteolin not only would exhibit enhanced antioxidant capability relative to ordinary collagen hydrogels, but its antioxidant power would also increase incrementally as additional luteolin was incorporated in the hydrogel solution. Hydrogels containing luteolin proved to contain superior antioxidant capacity, with significant enhancements observed as luteolin concentrations within the formula increased. The antioxidant performance of 120 μM hydrogels far surpassed the other formulations, providing a nearly 5-fold escalation in antioxidant capacity compared to 0 M. This behavior represents the ability of these materials to modulate the chemical environment around them by neutralizing ROS and hold promise to shield cells, both inside the of gel and around the wound site, from oxidative stress.

Encouraged by the high cytocompatibility and substantial antioxidant capabilities of the luteolin-collagen hydrogels, a final experiment explored during this work investigated the potential of the disclosed luteolin-hydrogels to protect cells against elevated, physiologically relevant H₂O₂ concentrations. H₂O₂ is prevalent ROS produced deliberately during wound healing and acts as a critical inter and extracellular signaling molecule, recruiting inflammatory and immune cells, among other functions, at injury onset.6 Effective REDOX signaling functions within a defined concentration of ROS in order for the cells and extracellular components to participate and manage such dangerous messengers and return to a state of REDOX homeostasis. Low and physiologically relevant levels of H₂O₂ have been reported at less than 100 μM, with 100 μM observed during inflammation49 Concentrations of as low as 500 μM can overwhelm the body's natural antioxidant mechanisms resulting in apoptosis in most systems.

Following 2-hour incubation in the presence of elevated H₂O₂ (300 M), NADFbs cultured in 3D within the luteolin gels resisted oxidative damage significantly better than controls. 120 μM luteolin gels provided more than double the protection of 0 μM gels against oxidative-induced metabolic impairment of embedded fibroblasts with strong statistical significance (p<0.0001). Antioxidant performance increased with luteolin concentration relative to control, yet 40 μM and 80 μM gels provided similar, and far more modest protection compared to 120 μM gels.

The importance of cellular protection against oxidative damage in a model ROS wound healing environment is twofold: 1. Cell-therapy payloads must be protected against oxidative damage at the wound site prior to hydrogel degradation and cell release or will otherwise be vulnerable to apoptosis, necrosis, and other forms of cellular damage. Thus, damaged cells will not effectively perform their secretory and regenerative functions to progress wound repair and will only contribute to sustaining inflammation and elevated ROS levels. 2. Hydrogels that can effectively neutralize ROS may be able to protect native cells at the wound bed from further oxidative damage and maximize their healing potential. The present study is the first in vitro step towards validating these concepts and formulating a potential luteolin-collagen construct.

### CONCLUSION

This study demonstrated the formulation, characterization, and biological evaluation of collagen hydrogels incorporating an antioxidant phytochemical, luteolin, and loaded with a therapeutic cell cargo for cutaneous wound healing and tissue engineering purposes. Luteolin-collagen hydrogels were found to be superior to typical collagen hydrogels over several therapeutic metrics. For instance, an increase in the concentration of luteolin within the gel formulation leads to stronger gels with higher gel modulus, which can potentially provide protection to the wound site against further injury. In addition, luteolin conferred a significant antioxidant advantage at higher concentrations. Furthermore, ROS challenge experiments confirmed the ability for the disclosed gels, especially 120 μM, to protect payload cells within the hydrogel from a hostile wound environment, ensuring the delivery of uncompromised cells to the wound site via natural degradation of the hydrogel. Cytotoxicity studies established adequate compatibility, though modestly decreasing in a dose-dependent manner, but can be balanced against the significant gain in antioxidant protection observed at higher concentrations of luteolin. Taken together, it is believed that luteolin-collagen hydrogels can not only modulate the chemical and cellular environment in and around the wound bed, potentially toward progressive healing events, but also protect a cellular payload contained within the hydrogel matrix from ROS damage prior to and during release at the site of injury. It is concluded that that luteolin-collagen hydrogels, especially high luteolin formulations like 120 μM demonstrate compelling potential as multifunctional tissue engineering materials.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Baolin, L.; et al. Topical Application of Luteolin Inhibits Scratching Behavior Associated with Allergic Cutaneous Reaction in Mice. Planta Med 2005, 71 (5), 424-428.
2. Boateng, J. S.; et al. Wound Healing Dressings and Drug Delivery Systems: A Review. J. Pharm. Sci. 2008, 97 (8), 2892-2923.
3. Comino-Sanz, I. M.; et al. Antioxidant Dressing Therapy versus Standard Wound Care in Chronic Wounds (the REOX Study): Study Protocol for a Randomized Controlled Trial. Trials 2020, 21 (1), 505.
4. Detwiler, A.; et al. Donor Age and Time in Culture Affect Dermal Fibroblast Contraction in an In Vitro Hydrogel Model. Tissue Eng Pt A 2022, 28 (19-20), 833-844.
5. Dhivya, S.; et al. Wound Dressings—a Review. Biomedicine 2015, 5 (4), 22.
6. Dhivya, S.; et al. Wound Dressings—a Review. Biomedicine 2015, 5 (4), 22.
7. Droge, W. Free Radicals in the Physiological Control of Cell Function. Physiol Rev 2002, 82 (1), 47-95.
8. Dunnill, C.; et al. Reactive Oxygen Species (ROS) and Wound Healing: The Functional Role of ROS and Emerging ROS-modulating Technologies for Augmentation of the Healing Process. Int Wound J 2017, 14 (1), 89-96.
9. Facchine, E. G.; et al. Shear-Dependent Structures of Flocculated Micro/Nanofibrillated Cellulose (MNFC) in Aqueous Suspensions. Biomacromolecules 2020, 21 (9), 3561-3570.
10. Farias, B. V.; et al. Linking Polymer Hydrophobicity and Molecular Interactions to Rheology and Tribology in Phospholipid-Containing Complex Gels. J Colloid Interf Sci 2021, 584, 134-144.
11. Fitzmaurice, S. D.; et al. Antioxidant Therapies for Wound Healing: A Clinical Guide to Currently Commercially Available Products. Skin Pharmacol Physi 2011, 24 (3), 113-126.
12. Gendrisch, F.; et al. Luteolin as a Modulator of Skin Aging and Inflammation. Biofactors 2021, 47 (2), 170-180.
13. Gorain, B.; et al. Advanced Drug Delivery Systems Containing Herbal Components for Wound Healing. Int J Pharmaceut 2022, 617, 121617.
14. Hampton, M. B.; et al. Dual Regulation of Caspase Activity by Hydrogen Peroxide: Implications for Apoptosis. Febs Lett 1997, 414 (3), 552-556.
15. Hao, Y.; et al. Luteolin Inhibits FcERI—and MRGPRX2-mediated Mast Cell Activation by Regulating Calcium Signaling Pathways. Phytother Res 2022, 36 (5), 2197-2206.
16. Heras, K. L.; et al. Chronic Wounds: Current Status, Available Strategies and Emerging Therapeutic Solutions. J Control Release 2020, 328, 532-550.
17. Huang, C.; et al. Anti-inflammatory Hydrogel Dressings and Skin Wound Healing. Clin Transl Medicine 2022, 12 (11), e1094.
18. Huizen, A. V. V.; et al. Reactive Oxygen Species Signaling Differentially Controls Wound Healing and Regeneration. Biorxiv 2022, 2022.04.05.487111.
19. Järbrink, K.; et al. Prevalence and Incidence of Chronic Wounds and Related Complications: A Protocol for a Systematic Review. Syst Rev 2016, 5 (1), 152.
20. KANAI, K.; et al. Therapeutic Anti-Inflammatory Effects of Luteolin on Endotoxin-Induced Uveitis in Lewis Rats. J Vet Medical Sci 2016, 78 (8), 1381-1384.
21. Kang, K. A.; et al. Luteolin Induces Apoptotic Cell Death via Antioxidant Activity in Human Colon Cancer Cells. Int J Oncol 2017, 51 (4), 1169-1178.
22. Kimata, M.; et al. Effects of Luteolin, Quercetin and Baicalein on Immunoglobulin E-mediated Mediator Release from Human Cultured Mast Cells. Clin Exp Allergy 2000, 30 (4), 501-508.
23. Kritas, S. K.; et al. Luteolin Inhibits Mast Cell-Mediated Allergic Inflammation. J Biol Reg Homeos Ag 2013, 27 (4), 955-959.
24. Lai, W.-F.; et al. Hydrogel-Based Materials for Delivery of Herbal Medicines. Acs Appl Mater Inter 2017, 9 (13), 11309-11320.
25. Lien, E. J.; et al. Quantitative Structure-Activity Relationship Analysis of Phenolic Antioxidants. Free Radical Bio Med 1999, 26 (3-4), 285-294.
26. Mora-Navarro, C.; et al. Monitoring Decellularization via Absorbance Spectroscopy during the Derivation of Extracellular Matrix Scaffolds. Biomed Mater 2022, 17 (1), 015008.
27. Mu, J.; et al. Luteolin Prevents UVB-Induced Skin Photoaging Damage by Modulating SIRT3/ROS/MAPK Signaling: An in Vitro and in Vivo Studies. Front Pharmacol 2021, 12, 728261.
28. Pachence, J. M. Collagen-based Devices for Soft Tissue Repair. J. Biomed. Mater. Res. 1996, 33 (1), 35-40.
29. Riha, S. M.; et al. Synergistic Effect of Biomaterial and Stem Cell for Skin Tissue Engineering in Cutaneous Wound Healing: A Concise Review. Polymers-basel 2021, 13 (10), 1546.
30. Sarker, P.; et al. High-Axial-Aspect Tannic Acid Microparticles Facilitate Gelation and Injectability of Collagen-Based Hydrogels. Biomacromolecules 2022, 23 (11), 4696-4708.
31. Schieber, M.; et al. ROS Function in Redox Signaling and Oxidative Stress. Curr Biol 2014, 24 (10), R453-R462.
32. Schindelin, J.; et al. Fiji: An Open-Source Platform for Biological-Image Analysis. Nat Methods 2012, 9 (7), 676-682.
33. Schomberg, J.; et al. Luteolin Inhibits Melanoma Growth in Vitro and in Vivo via Regulating ECM and Oncogenic Pathways but Not ROS. Biochem Pharmacol 2020, 177, 114025.
34. Sen, C. K. Human Wound and Its Burden: Updated 2020 Compendium of Estimates. Adv Wound Care 2021, 10 (5), 281-292.
35. Sharma, A.; et al. Herbal Bioactive-Based Drug Delivery Systems. 2022, 311-330.
36. Singh, H.; et al. Curcumin in Decellularized Goat Small Intestine Submucosa for Wound Healing and Skin Tissue Engineering. J Biomed Mater Res Part B Appl Biomaterials 2021.
37. Sivaraj, D.; et al. Hydrogel Scaffolds to Deliver Cell Therapies for Wound Healing. Frontiers Bioeng Biotechnology 2021, 09, 660145.
38. Song, X.; et al. Properties of Cell-Compatible Poly(Vinyl Alcohol) Hydrogels Cross-Linked with Hydrophobic Luteolin. Acs Appl Polym Mater 2021, 3 (6), 3019-3027.
39. Vesper, E. O.; et al. Even with Rehydration, Preservation in Ethanol Influences the Mechanical Properties of Bone and How Bone Responds to Experimental Manipulation. Bone 2017, 97, 49-53.
40. Vliet, A. van der; et al. Hydrogen Peroxide as a Damage Signal in Tissue Injury and Inflammation: Murderer, Mediator, or Messenger? J. Cell. Biochem. 2014, 115 (3), 427-435.
41. Wang, G.; et al. The Initiation of Oxidative Stress and Therapeutic Strategies in Wound Healing. Biomed Pharmacother 2023, 157, 114004.
42. Wang, J.; et al. Protective Effect of Luteolin on Atopic Dermatitis Murine Model via IgE Mediated Immune Response. Israel J Plant Sci 2021, 1-8.
43. Wang, R. M.; et al. Myocardial Matrix Hydrogel Acts as a Reactive Oxygen Species Scavenger and Supports a Proliferative Microenvironment for Cardiomyocytes. Acta Biomater 2022, 152, 47-59.
44. Wang, Y.; et al. Collagen-Based Biomaterials for Tissue Engineering. Acs Biomater Sci Eng 2023, 9 (3), 1132-1150.
45. Weng, Z.; et al. Luteolin Inhibits Human Keratinocyte Activation and Decreases NF-KB Induction That Is Increased in Psoriatic Skin. Plos One 2014, 9 (2), e90739.
46. Wölfle, U.; et al. UVB-Induced DNA Damage, Generation of Reactive Oxygen Species, and Inflammation Are Effectively Attenuated by the Flavonoid Luteolin in Vitro and in Vivo. Free Radical Bio Med 2011, 50 (9), 1081-1093.
47. Xu, Z.; et al. Advances and Impact of Antioxidant Hydrogel in Chronic Wound Healing. Adv Healthc Mater 2020, 9 (5), 1901502.
48. Yang, X.; et al. Spectroscopy Study on the Interaction of Quercetin with Collagen. J Agr Food Chem 2009, 57 (9), 3431-3435.
49. Young, A. T.; et al. Smart Materials for Tissue Engineering: Applications. Smart Mater Ser 2017, 642-670.
50. Zhu, J.; et al. Collagen I Self-Assembly: Revealing the Developing Structures That Generate Turbidity. Biophys J 2014, 106 (8), 1822-1831.
51. Freytes, D. O. et al., US 2023/0181796, Jun. 15, 2023.

What is claimed is:

1. A composition comprising a collagen hydrogel and a flavonoid, wherein at least a portion of the collagen hydrogel is crosslinked by the flavonoid.

2. The composition of claim 1, wherein the flavonoid comprises a flavonol, flavone, anthocyanidin, isoflavonoid, neoflavonoid, an anthoxanthin, a derivative thereof, or any combination thereof.

3. The composition of claim 2, wherein the flavonol comprises catechin, gallocatechin, catechin 3-gallate, gallocatechin 3-gallate, epicatechin, epigallocatechin, epicatechin 3-gallate, epigallocatechin 3-gallate, leucoanthocyanidin, a derivative thereof, or any combination thereof.

4. The composition of claim 2, wherein the neoflavonoid comprises calophylloid,
dalbergichromene, coutareagenin, dalbergin, nivetin, a derivative thereof, or any combination thereof.

5. The composition of claim 2, wherein the anthoxanthin comprises quercetin, betaxanthin, canthaxanthin, a derivative thereof, or any combination thereof.

6. The composition of claim 2, wherein the flavone comprises primuletin, chrysin, tetochrysin, primetin, apigenin, acacetin, genkwanin, echoidinin, baicalein, oroxylin A, negletein, norwogonin, wogonin, liquiritigenin, naringenin, geraldone, tithonine, luteolin, 6-hydroxyluteolin, chrysoeriol, diosmetin, pillion, velutin, norartocarpetin, artocarpetin, scutellarein, hispidulin, sorbifolin, pectolinarigenin, cirsimaritin, mikanin, isoscutellarein, zapotinin, zapotin, cerrosillin, alnetin, tricetin, tricin, corymbosin, nepetin, pedalitin, nodifloretin, jaceosidin, cirsiliol, eupatorine, sinensetin, hypolaetin, onopordin, wightin, nevadensin, xanthomicrol, tangeretin, serpyllin, sudachitin, acerosin, hymenoxin, gardenin D, nobiletin, scaposin, a derivative thereof, or any combination thereof.

7. The composition of claim 1, wherein the collagen hydrogel comprises type I collagen, type II collagen, type III collagen, type IV collagen, type V collagen, type VI collagen, type VII collagen, type VIII collagen, type IX collagen, type XI collagen, type XXI collagen, or any combination thereof.

8. The composition of claim 1, wherein the collagen hydrogel comprises bovine collagen, porcine collagen, chicken collagen, turkey collagen, egg collagen, fish collagen, recombinant collagen produced by bacterial fermentation, extracellular matrix (ECM)-derived collagen, or any combination thereof.

9. The composition of claim 1, wherein the composition is cytocompatible.

10. The composition of claim 1, wherein the composition protects cells from oxidative damage.

11. The composition of claim 1, wherein the composition comprises from about 5 μM to about 400 μM of the flavonoid.

12. The composition of claim 1, wherein the collagen hydrogel has an average pore size of from about 0.5 μm to about 10 μm.

13. A method for preparing a collagen hydrogel, the method comprising:
(a) contacting a pre-gel solution of collagen with a solution of flavonoid in a solvent to create a first admixture;
(b) casting the first admixture in a container; and
(c) incubating the first admixture to produce the hydrogel.

14. The method of claim 13, wherein the pre-gel solution of collagen has a concentration of from about 0.5 to about 15 mg/mL.

15. The method of claim 13, wherein the solvent comprises ethanol, dimethyl sulfoxide (DMSO), dimethylformamide (DMF), tetrahydrofuran (THF), propanol, lipid nanoparticles, methanol, ethyl acetate, acetonitrile, or any combination thereof.

16. A pharmaceutical composition comprising the composition of claim 1.

17. An article comprising the pharmaceutical composition of claim 16.

18. The article of claim 17, wherein the article comprises a bandage or a wound dressing.

19. A method for promoting or enhancing wound healing, the method comprising applying the article according to claim 18 to a wound.

20. The method of claim 19, wherein the wound comprises a diabetic wound, a burn, or a surgical incision.
